(19) United States
(12) Patent Application Publication
(10) Pub. No.: US 2022/0387519 A1
(43) Pub. Date: Dec. 8, 2022

(54) Method For Treating Cancer With Xenogeneic Tissue Cell Composition Of Similar Or Same Histological Type

(71) Applicant: China Medical University, Taichung City (TW)

(72) Inventors: Chih-Rong Shyr, Taichung City (TW); Chi-Ping Huang, Taichung City (TW); Chih-Chun Chang, Taichung City (TW); Chun-Chie Wu, Taichung City (TW)

(73) Assignee: China Medical University, Taichung City (TW)

(21) Appl. No.: 17/776,284

(22) PCT Filed: Nov. 13, 2020
(86) PCT No.: PCT/CN2020/128782

§ 371 Date: May 12, 2022

Related U.S. Application Data
(60) Provisional application No. 62/934,633, filed on Nov. 13, 2019.

(51) Int. Cl. A61K 35/44; A61K 35/28; A61K 47/36; A61K 38/00; A61K 9/00; A61P 35/00

(52) U.S. Cl. CPC A61K 35/44; A61K 35/28; A61K 47/36; A61K 38/005; A61K 9/0019; A61P 35/00

(57) ABSTRACT

A method for treating a cancer of similar or same histological type is provided. The method includes administering a xenogeneic tissue cell composition to a subject in need for a treatment of the cancer of similar or same histological type. The xenogeneic tissue cell composition does not include a tumor cell, and the xenogeneic tissue cell composition is administered to the cancer of the similar or same histological type by an intralesional route.

### RELATED APPLICATIONS

This application is a National Stage of International application No. PCT/CN2020/128782, filed Nov. 13, 2020, which claims the benefits of priority of U.S. Provisional Application No. 62/934,633, filed on Nov. 13, 2019, the content of which are incorporated herein by reference.

### BACKGROUND

### Technical Field

The present disclosure relates to a use of a xenogeneic tissue cell composition. More particularly, the present disclosure relates to a use of a xenogeneic tissue cell composition for treating a cancer.

### Description of Related Art

Cancer, also known as malignancy, is a state of abnormal proliferation of cells, and these proliferating cells may invade other parts of the body as a disease caused by a malfunction in the control of cell division and proliferation. The number of people suffering from cancer worldwide has a growing trend. Cancer is one of the top ten causes of death for the Chinese people and has been the top ten causes of death for twenty-seven consecutive years. Although current cancer therapies, such as surgery, radiation therapy, chemotherapy and targeted therapy, are effective, they come with collateral damage that damages healthy tissue and can cause many complications and adverse effects. Even more desperate, even with many treatment options, for certain advanced cancer patients with a 5-year survival rate of less than 10%, the cancer mortality rate has declined only slightly and the results are dismal. Regardless of the broad range of therapeutic strategies, cancer cells can find ways to evade attack and develop a new form to thrive through complex interconnected genetic and signaling pathways.

Cancer immunotherapy has recently emerged as the fourth major cancer treatment, both innate and adaptive immunity are involved in cancer immune surveillance. Thus specific innate and adaptive immune cell types such as T cells, B cells and natural killer T cells, dendritic cells, effector molecules and regulatory pathways work together to inhibit tumor formation. Cancer immunotherapy activates the host immune system with immunomodulatory agents or genetically engineered T cells to eradicate tumors that evade the immune system with long-lasting durable responses.

Different types of infiltrating immune cells have different effects on tumor progression, depending on the type of cancer and the specificity of the infiltrating immune cells. Although immunomodulatory antibodies against cytotoxic T-lymphocyte-associated protein 4 (CTLA4) and programmed death-1/programmed death-ligand 1 (PD-1/PD-L1) have excellent therapeutic effects in clinical practice, only a subset of patients can show durable responses, suggesting that a broader view of cancer immunity is needed in the treatment of cancer.

### SUMMARY

According to one aspect of the present disclosure is to provide a method for treating a cancer of similar or same histological type. The method includes administering a xenogeneic tissue cell composition to a subject in need for a treatment of the cancer of similar or same histological type, wherein the xenogeneic tissue cell composition does not include a tumor cell, and the xenogeneic tissue cell composition is administered to the cancer of similar or same histological type by an intralesional route.

### BRIEF DESCRIPTION OF THE DRAWINGS

In order to make the above and other objectives, features, advantages and embodiments of the present disclosure more comprehensible, the description of the accompanying drawings is as follows:

FIGS. 1A, 1B, 1C are schematic diagrams showing the treatment of cancers of similar or same histological type with xenogeneic tissue cell compositions of the present disclosure;

FIGS. 2A and 2B show the analysis results of cell characteristics of a xenogeneic mammary cell composition isolated from porcine mammary gland tissue;

FIGS. 3A, 3B, 3C show the analysis results of the effect of the xenogeneic mammary cell composition in the treatment of breast cancer;

FIGS. 4A and 4B show the analysis results of cell characteristics of a xenogeneic renal cell composition isolated from porcine kidney tissue;

FIGS. 5A, 5B, 5C show the analysis results of the effect of the xenogeneic renal cell composition in the treatment of kidney cancer;

FIGS. 6A and 6B show the analysis results of cell characteristics of a xenogeneic liver cell composition isolated from porcine liver tissue;

FIG. 7 shows the analysis results of the effect of the xenogeneic liver cell composition in the treatment of liver cancer;

FIGS. 8A and 8B show the analysis results of cell characteristics of a xenogeneic urothelial cell composition isolated from porcine;

FIGS. 9A, 9B, 9C show the analysis results of the effect of the xenogeneic urothelial cell composition in the treatment of bladder cancer;

FIGS. 10A and 10B show the analysis results of cell characteristics of a xenogeneic pancreatic cell composition isolated from porcine pancreatic tissue; and

FIG. 11 shows the analysis results of the effect of the xenogeneic pancreatic cell composition in the treatment of pancreatic cancer.

### DETAILED DESCRIPTION

The present disclosure provides a novel method for treating cancer by using a xenogeneic tissue cell composition with expansion potential isolated and expanded from a xenogeneic source, and using the expanded xenogeneic tissue cell composition to treat cancers of similar or same histological type. Specifically, the present disclosure provides a novel use of the xenogeneic tissue cell composition administered by an intralesional route to tumor sites of similar or same histological type to repair damaged tissue and restore the immune system to treat cancer. The xenogeneic tissue cell composition can be used as an immune agent to enhance the human immune system to eradicate tumor cells, providing a new treatment option for cancer immunotherapy. Therefore, the present disclosure is a method for treating cancer that utilizes the rejection immune response of the host immune system to xenogeneic cells to induce anti-tumor immunity.

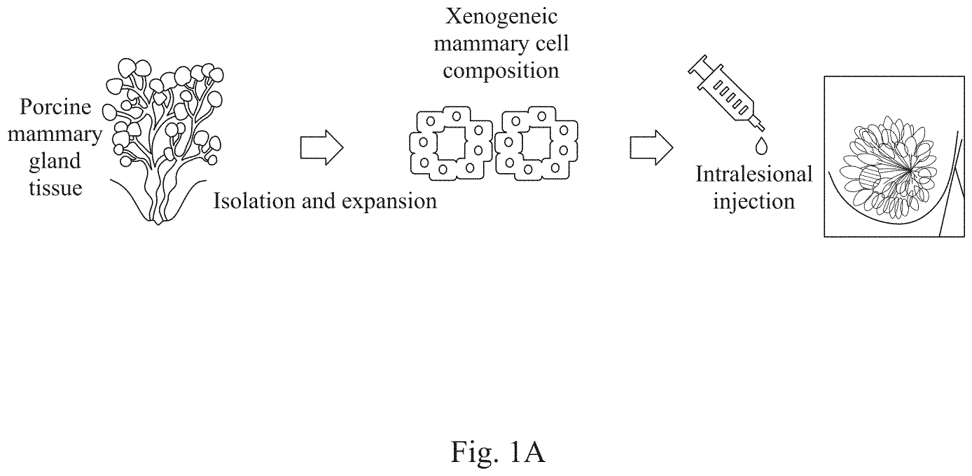

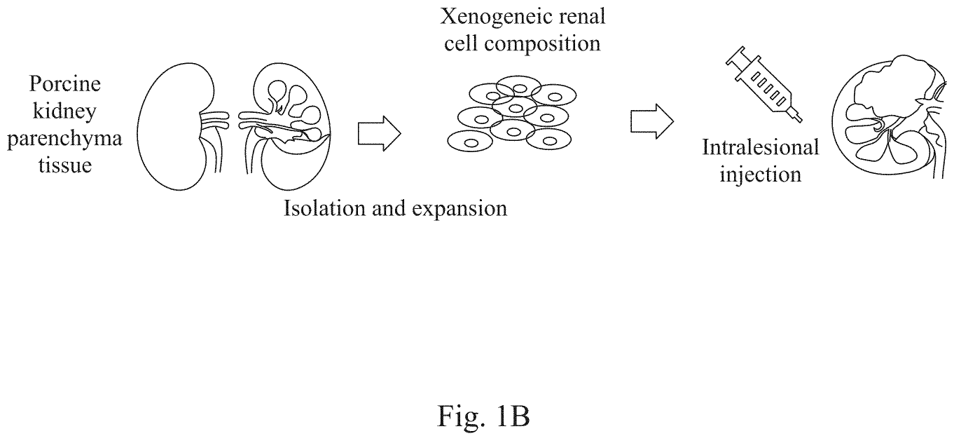

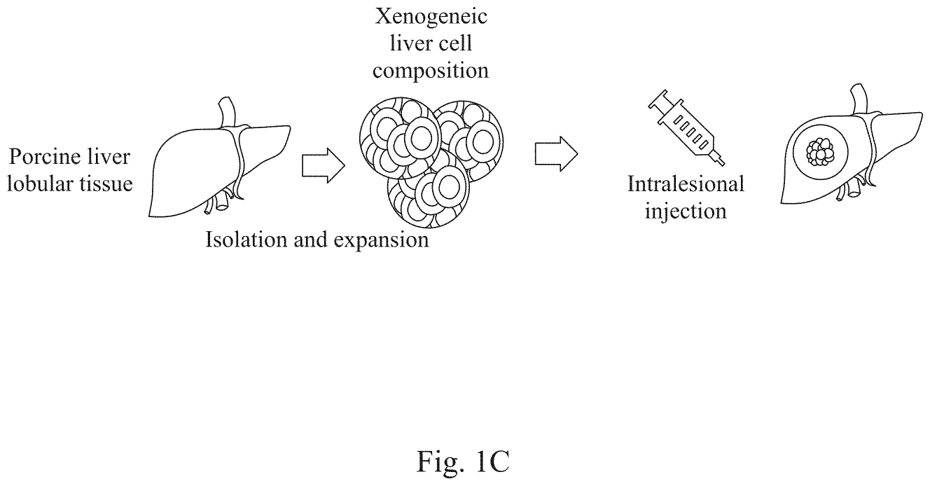

Please refer to FIGS. 1A to 1C, which are schematic diagrams showing the treatment of cancers of similar or same histological type with xenogeneic tissue cell compositions of the present disclosure. FIG. 1A is a schematic diagram showing the treatment of breast cancer with xenogeneic mammary epithelial cells, FIG. 1B is a schematic diagram showing the treatment of kidney cancer with xenogeneic renal cells, and FIG. 1C is a schematic diagram showing the treatment of liver cancer with xenogeneic liver cells. However, the types of cancers that can be treated with the xenogeneic tissue cell composition of the present disclosure are not limited to those exemplified in FIGS. 1A to 1C.

As shown in FIG. 1A, when the xenogeneic tissue cell composition of the present disclosure is used to treat breast cancer, the xenogeneic mammary cell composition can be isolated from a xenogeneic breast tissue, such as porcine mammary gland, which includes xenogeneic mammary stem cells, xenogeneic mammary progenitor cells, xenogeneic mammary gland precursors and xenogeneic mammary epithelial cells. The isolated xenogeneic tissue cell composition is expanded, and then the expanded xenogeneic mammary cell composition is injected into the lesion site of breast cancer by the intralesional injection, and the immune system's rejection of the xenogeneic mammary cell composition is used to trigger anti-tumor immunity to achieve the effect of treating breast cancer.

As shown in FIG. 1B, when the xenogeneic tissue cell composition of the present disclosure is used to treat kidney cancer, the xenogeneic renal cell composition can be isolated from a xenogeneic kidney tissue, such as porcine kidney parenchyma tissue, which includes xenogeneic renal stem cells, xenogeneic renal progenitor cells, xenogeneic renal precursors and xenogeneic renal epithelial cells. The isolated xenogeneic renal cell composition is expanded, and then the expanded xenogeneic renal cell composition is injected into the lesion site of kidney cancer by the intralesional injection, and the immune system's rejection of the xenogeneic renal cell composition is used to trigger anti-tumor immunity to achieve the effect of treating kidney cancer.

As shown in FIG. 1C, when the xenogeneic tissue cell composition of the present disclosure is used to treat liver cancer, the xenogeneic liver cell composition can be isolated from a xenogeneic liver tissue, such as porcine liver lobular tissue, which includes xenogeneic liver stem cells, xenogeneic liver progenitor cells, xenogeneic liver precursors and xenogeneic liver cells. The isolated xenogeneic liver cell composition is expanded, and then the expanded xenogeneic liver cell composition is injected into the lesion site of liver cancer by the intralesional injection, and the immune system's rejection of the xenogeneic liver cell composition is used to trigger anti-tumor immunity to achieve the effect of treating liver cancer.

According to the present disclosure, the intralesional route can include an intratumoral administration and a peritumoral administration. The xenogeneic tissue cell composition of the present disclosure can be administered with the assistance of an imaging system (such as an ultrasound apparatus, an endoscope, a computed tomography system, an X-ray machine, a nuclear magnetic resonance apparatus, a fluoroscopy, or a positron emission tomography apparatus) to guide the injection needle to target the location of the tumor lesion while the xenogeneic tissue cell composition can be injected into the tumor area.

According to the present disclosure, the xenogeneic tissue cell composition can be isolated from a mammal, and the mammal can be a human, a pig, a dog, a cat, a cow, a horse, a donkey, a deer, a goat, a sheep, a rabbit, a mouse, a rat, a guinea pig, a monkey and/or any other mammals used as livestock or as pets.

According to the present disclosure, the xenogeneic tissue cell composition includes xenogeneic tissue-specific stem cells, xenogeneic tissue progenitor cells, xenogeneic tissue precursors and xenogeneic tissue mature cells. Further, the xenogeneic tissue cell composition can be in a solution containing an extracellular matrix molecule and a polysaccharide. Thereby, the xenogeneic tissue cell composition can be contacted with an effective amount of growth factors, extracellular matrix, and signaling pathway modulators to maintain the xenogeneic tissue-specific stem cell population and the xenogeneic tissue progenitor cell population, and to expand the xenogeneic tissue precursors and xenogeneic tissue mature cells to increase total cell number.

According to the present disclosure, the xenogeneic tissue precursor is selected from the group consisting of xenogeneic tissue-specific stem cells, xenogeneic tissue progenitor cells, xenogeneic tissue precursor cells and combinations thereof. The xenogeneic tissue cell composition includes at least 50% and 90% of the xenogeneic tissue-specific stem cells/xenogeneic tissue progenitor cells, and the isolated xenogeneic tissue-specific stem cells/xenogeneic tissue progenitor cells or their populations express corresponding marker genes thereof.

According to the present disclosure, the cellular phenotype of the expanded xenogeneic tissue cell composition can be determined by assessing markers, the expression of which can be assessed by various methods known in the art. The presence of the marker can be determined in terms of DNA, RNA or polypeptide expression. For example, detecting the polypeptide expression of the marker gene can be included, and the polypeptide expression includes the presence or absence of the marker gene polypeptide sequence, which can be detected by various techniques known in the art, including by sequencing and/or binding to specific ligands (e.g., antibodies). For example, polypeptide expression can be assessed by methods including, but not limited to, immunostaining, flow cytometry analysis, or Western blotting. For detecting the RNA expression of a marker gene (such as p63, CD33, CD44, CD133, ALDH or a combination thereof) in the xenogeneic tissue cell composition, and the RNA expression includes the presence of RNA sequence, the presence of RNA splicing or processing or the presence of a certain amount of RNA, which can be detected by various techniques known in the art, including by sequencing all or part of the marker gene RNA, or by selective hybridization or selective amplification of all or part of the RNA. The aforementioned method is known in the art, and details are not described herein again.

“Treating” “treat” or “treatment” refers to administering the xenogeneic tissue cell composition of present disclosure to a subject in need, such as a cancer patient.

An “effective amount” refers to an amount of the xenogeneic tissue cell composition effective to “treat” a disease or disorder in a subject. The effective amount is to some extent related to the biological or medical response of the tissue, system, animal or human to whom it is administered, for example, when administered, it is sufficient to prevent the development of one or more diseases or conditions or to alleviate the symptoms of one or more conditions or conditions being treated to a certain extent. A therapeutically effective amount will vary depending on the disease and its severity, as well as the age and weight of the mammal to be treated.

“Ameliorate” refers to decreasing, suppressing, attenuating, diminishing, arresting or stabilizing the development or progression of a disease or a symptom thereof.

“Cancer” refers to a physiological condition in a mammal characterized by a disorder of cell growth. A “tumor” includes one or more cancer cells. Examples of cancer include, but are not limited to, carcinoma, lymphoma, blastoma, sarcoma, and leukemia or lymphoid malignancies. More specific examples of such cancers include squamous cell carcinoma (e.g., epithelial squamous cell carcinoma), lung cancer including small cell lung cancer, non-small cell lung cancer (NSCLC), lung adenoma, and lung squamous cell carcinoma, peritoneal cancer, hepatocellular carcinoma, gastric cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine cancer, salivary gland cancer, kidney cancer, prostate cancer, vulvar cancer, thyroid cancer, anal cancer, penile cancer, and head and neck cancer.

The present disclosure can treat virtually any type of cancer, including acute myeloid leukemia, adrenocortical carcinoma, AIDS-related cancer, AIDS-related lymphoma, anal cancer, appendix cancer, astrocytoma, basal cell carcinoma, extrahepatic bile duct cancer, bladder cancer, bone cancer, osteosarcoma/malignant fibrous histiocytoma, brain stem glioma, brain tumor, cerebellar astrocytoma, cerebral astrocytoma/malignantglioma, ependymoma, medulloblastoma, supratentorial primitive neuroectodermal tumor, visual pathway and hypothalamic glioma, breast cancer, bronchial adenoma/carcinoid, childhood carcinoid tumor, gastrointestinal carcinoid, unknown primary carcinoma, primary central nervous system lymphoma, childhood cerebellar astrocytoma, childhood cerebral astrocytoma/malignant glioma, cervical cancer, childhood cancer, chronic myeloproliferative disease cancer, colon cancer, cutaneous T-cell lymphoma, desmoplastic small round cell tumor, endometrial cancer, Esophageal cancer, Ewing's sarcoma, childhood extracranial germ cell tumor, eye cancer, retinoblastoma, gallbladder cancer, gastric cancer, gastrointestinal stromal tumor (GIST), germ cell tumor (extracranial, extragonadal, or ovarian), gestational trophoblastic tumor, childhood cerebral astrocytoma glioma, childhood visual pathway and hypothalamic glioma, gastric carcinoid, hairy cell leukemia, head and neck cancer, hepatocellular (liver) cancer, hypopharyngeal cancer, islet cell carcinoma (endocrine pancreas), Kaposi sarcoma, kidney cancer, laryngeal cancer, leukemia, acute lymphocytic leukemia, acute myeloid leukemia (also called acute myelogenous leukemia), chronic lymphocytic leukemia, chronic myelogenous leukemia (also called chronic myeloid leukemia), lip cancer, oral cancer, liposarcoma, liver cancer (primary), non-small cell lung cancer, small cell lung cancer, lymphomas, Burkitt lymphoma, Hodgkin lymphoma, non-Hodgkin lymphoma (an old classification of all lymphomas except Hodgkin's), macroglobulinemia, Waldenström macroglobulinemia, malignant fibrous histiocytoma of bone/osteosarcoma, childhood medulloblastoma, melanoma, intraocular (eye) melanoma, Merkel cell carcinoma, malignant mesothelioma in adult, childhood mesothelioma, metastatic squamous neck cancer with occult primary, childhood multiple endocrine neoplasia, multiple myeloma/plasma cell neoplasm, mycosis fungoides, myelodysplastic syndrome, myelodysplastic/myeloproliferative disease, chronic myeloid leukemia, adult acute myeloid leukemia, childhood acute myeloma leukemia, multiple myeloma (cancer of the bone-marrow), nasal cavity and paranasal sinus cancer, nasopharyngeal carcinoma, neuroblastoma, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, parathyroid cancer, penile cancer, pharyngeal cancer, pheochromocytoma, pineal astrocytoma, pineal germinoma, childhood pineoblastoma and supratentorial primitive neuroectodermal tumor, pituitary adenoma, plasma cell neoplasia/multiple myeloma, pleuropulmonary blastoma, prostate cancer, rectal cancer, renal cell carcinoma, transitional cell cancer of renal pelvis and ureter, childhood rhabdomyosarcoma, salivary gland cancer, sarcoma, Ewing family of tumor, Kaposi's sarcoma, soft tissue sarcoma, uterine sarcoma, Sezary syndrome, skin cancer (non-melanoma), skin cancer (melanoma), small intestine cancer, squamous cell carcinoma, childhood supratentorial primitive neuroectodermal tumor, testicular cancer, thymoma, childhood thymoma, thymic carcinoma, thyroid cancer, childhood thyroid cancer, cancer of unknown primary site in adult, cancer of unknown primary site in childhood, urethral cancer, endometrial cancer, vaginal cancer, vulvar cancer, and childhood Wilms tumor.

The xenogeneic tissue cell composition of the present disclosure can be administered in combination with at least one other therapeutic agent, for example, a chemotherapy drug, a targeted therapy drug, an antibody drug, an immunomodulatory agent, or a combination thereof.

The chemotherapy drug is selected from the group consisting of an alkylating agent, a nitrosourea agent, an antimetabolite, an antitumor antibiotic, an alkaloid derived from a plant, a topoisomerase inhibitor, a hormone therapy medicine, a hormone antagonist, an aromatase inhibitor, a P-glycoprotein inhibitor and a platinum complex derivative. Examples of the chemotherapy drug include, but are not limited to, Abiraterone Acetate, Afatinib, Aldesleukin, Alemtuzumab, Alitretinoin, Altretamine, Amifostine, Aminoglutethimide, Anagrelide, Anastrozole, Arsenic Trioxide, Asparaginase, Azacitidine, Azathioprine, Bendamustine, Bevacizumab, Bexarotine, Bicalutamide, Bleomycin, Bortezomib, Busulfan, Capecitabine, Carboplatin, Carmustine, Cetuximab, Chlorambucil, Cisplatin, Cladribine, Crizotinib, Cyclophosphamide, Cytarabine, Dacarbazine, Dactinomycin, Dasatinib, Daunorubicin, Denileukin diftitox, Decitabine, Docetaxel, Dexamethasone, Doxifluridine, Doxorubicin, Epirubicin, Epoetin Alpha, Epothilone, Erlotinib, Estramustine, Etinostat, Etoposide, Everolimus, Exemestane, Filgrastim, Floxuridine, Fludarabine, Fluorouracil, Fluoxymesterone, Flutamide, folated linked alkaloids, Gefitinib, Gemcitabine, Gemtuzumab ozogamicin, GM-CT-01, Goserelin, Hydroxyureas, Ibritumomab, Idarubicin, Ifosfamide, Imatinib, Interferon alpha, Interferon beta, Irinotecan, Ixabepilone, Lapatinib, Leucovorin, Leuprolide, Lenalidomide, Letrozole, Lomustine, Mechlorethamine, Megestrol, Melphalan, Mercaptopurine, Methotrexate, Mitomycin, Mitoxantrone, Nelarabine, Nilotinib, Nilutamide, Octreotide, Ofatumumab, Oprelvekin, Oxaliplatin, Paclitaxel, Panitumumab, Pemetrexed, Pentostatin, polysaccharide galectin inhibitors, Procarbazine, Raloxifene, Retinoic acids, Rituximab, Romiplostim, Sargramostim, Sorafenib, Streptozocin, Sunitinib, Tamoxifen, Temsirolimus, Temozolamide, Teniposide, Thalidomide, Thioguanine, Thiotepa, Tioguanine, Topotecan, Toremifene, Tositumomab, Trametinib, Trastuzumab, Tretinoin, Valrubicin, VEGF inhibitors and traps, Vinblastine, Vincristine, Vindesine, Vinorelbine, Vintafolide (EC145), Vorinostat, a salt thereof, or any combination of the foregoing.

The targeted therapy drug can be a small molecule, a small molecule conjugate or a monoclonal antibody, which can be selected from a tyrosine kinase inhibitor, a mitogen-activated protein kinase inhibitor, a JAK kinase inhibitor, an anaplastic lymphoma kinase inhibitor, a B-cell lymphoma-2 inhibitor, a poly ADP ribose polymerase inhibitor, a selective estrogen receptor modulator, a phosphatidylinositol trikinase inhibitor, a Braf inhibitor, a cyclin-dependent kinase inhibitor and a heat shock protein 90 inhibitor. Examples of targeted therapy drugs include, but are not limited to, Imatinib Mesylate, Gefitinib, Erlotinib, Bortezomib, Tamoxifen, Tofacitinib, Crizotinib, ABT-263, Gossypol, Olapa, Perifoxine, Apatinib, AN-152, (AEZS-108) Doxorubicin conjugated [D-Lys(6)]-LHRH, Vemurafenib, Dabrafenib, LGX818, Trametinib, MEK162, PD-0332991, LEE011, Salinomycin, Vintafolide, Rituximab, Trastuzumab, Cetuximab, Bevacizumab, or any combination of the foregoing.

The antibody drug can be selected from an antibody and an antibody drug complex. Examples of antibody drugs include, but are not limited to, Alemtuzumab (Campath), Bevacizumab (AVASTIN®, Genentech), Cetuximab (ERBITUX®, Imclone), Parimumab (VECTIBIX®, Amgen), Rituximab (RITUXAN®, Genentech/Biogen Idee), Pertuzumab (OMNITARG™, 2C4, Genentech), Trostuzumab (HERCEPTIN®, Genentech), Tositumomab (Bexxar, Corixia), and antibody drug conjugate, Gemtuzumab ozogamicin (MYLOTARG®, Wyeth) or any combination thereof.

The immunomodulator can be selected from a cytokine and an immune checkpoint inhibitor. Examples of immunomodulator include, but are not limited to, TLR agonist (TLR1, TLR2, TLR3, TLR4, TLR5, TLR6, TLR7, TLR8, TLR9, TLR10, TLR11 agonist), type 1 interferons (α-interferon or β-interferon as appropriate), CD40 agonist, IL-6 antagonist, TNF antagonist, cytotoxic T lymphocyte-associated antigen 4 (CTLA-4) inhibitor [such as Ipilimumab (Yervoy®)], programmed death-1 (PD-1) inhibitor [such as Pembrolizumab (Keytruda®) or Nivolumab (Opdivo®)] and programmed death-ligand 1 (PD-L1) inhibitor [such as Atezolizumab (Tecentriq®)].

The following specific examples are used to further illustrate the present disclosure, in order to benefit the person having ordinary skill in the art, and can fully utilize and practice the present disclosure without excessive interpretation. These examples should not be regarded as limiting the scope of the present disclosure, but is used to illustrate how to implement the materials and methods of the present disclosure.

I. The Effect of Xenogeneic Tissue Cell Composition in the Treatment of Breast Cancer

To demonstrate the anti-breast cancer effect of the xenogeneic tissue cell composition administered by the intralesional route, the xenogeneic mammary cell composition isolated from porcine mammary gland tissue and expanded, and an immunocompetent orthotopic 4T1 breast tumor mouse model is used to explore the efficacy thereof.

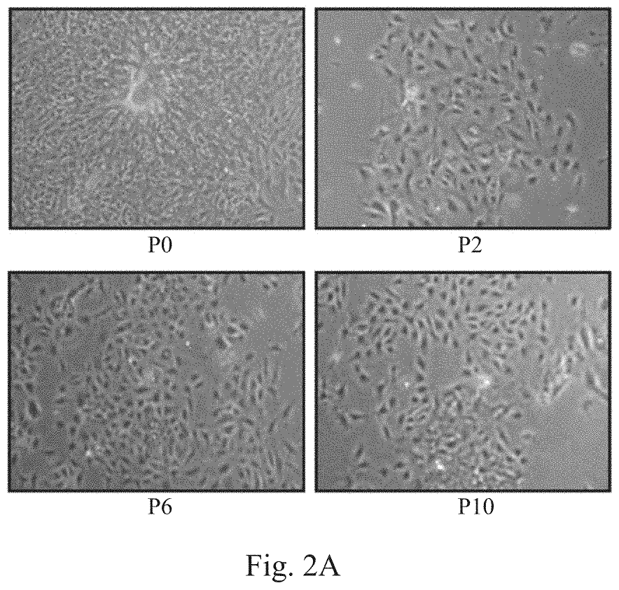

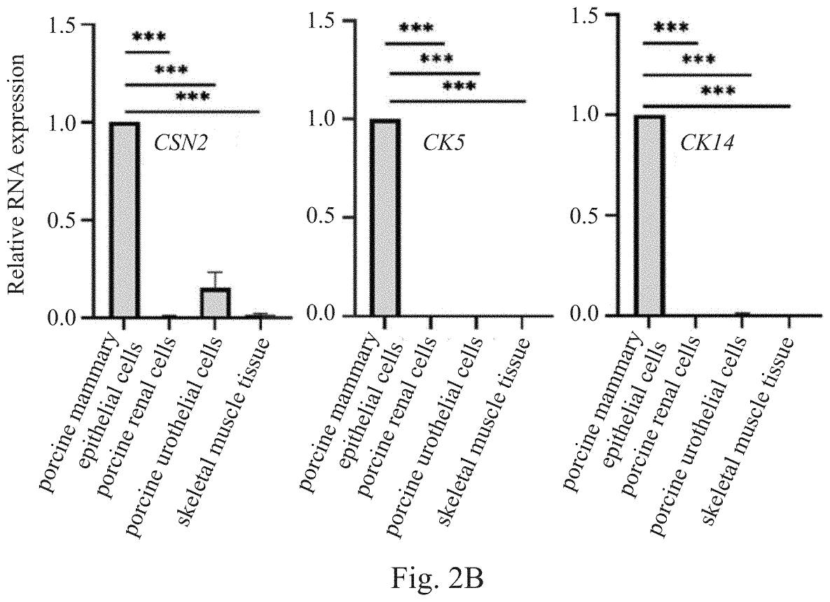

In the experiment, the isolation and expansion of the xenogeneic mammary cell composition is performed first, and the cell characteristics of the isolated xenogeneic mammary cell composition are analyzed. Please refer to FIGS. 2A and 2B, which show the analysis results of cell characteristics of the xenogeneic mammary cell composition isolated from porcine mammary gland tissue. FIG. 2A shows photomicrographs of porcine mammary epithelial cells of different passages, and FIG. 2B is the analysis result of expression analysis of related genes in porcine mammary epithelial cells.

The results in FIG. 2A show that the isolated xenogeneic mammary cell composition has the morphology of porcine mammary gland epithelial cells. The results in FIG. 2B show that compared with porcine renal cells, porcine urothelial cells and skeletal muscle tissue, only porcine mammary epithelial cells abundantly express mammary epithelial cell-related genes such as CSN2 gene, CK5 gene and CK14 gene, while other cells express none or a small amount of the mammary epithelial cell-related genes. Statistical analysis in FIG. 2B was performed by one-way ANOVA, data are presented as mean±SD, and *** indicates p<0.001.

A syngeneic animal model that is similar to breast tumor and has a complete immune system is used to further evaluate the anti-breast cancer effect of the xenogeneic mammary cell composition in vivo. Therefore, an orthotopic 4T1 breast tumor mouse model was established first. The 4T1 breast cancer cell line (1×10⁶) derived from BALB/c mice was injected into the mammary fat pad of immunocompetent BALB/c mice, and the implanted 4T1 breast cancer cell line developed into tumors. The model was established when the tumor size reached to 50-100 mm³, and subsequent treatment was performed. There were 4 groups in the experiment, untreated control group, the test group 1 treated with the xenogeneic mammary cell composition, the test group 2 treated with chemotherapy drug, and the test group 3 treated with both the xenogeneic mammary cell composition and the chemotherapy drug, wherein the chemotherapy drug used in this experiment was Gemcitabine. The xenogeneic mammary cell composition treated in test group 1 and test group 3 was intratumorally injected with 1×10⁶ xenogeneic mammary cell composition on day 1 of the experiment. The chemotherapy drug treated in test group 2 and test group 3 was intraperitoneally injected Gemcitabine at 60 mg/kg on days 2, 7 and 14 of the experiment, and the treatment time was 3 weeks. The tumor growth was assessed by measuring tumor volumes twice a week with a caliper and the volume is calculated using the formula: π/6×length×width². Tumor tissue was harvested and weighed when the mice died or reached the experiment endpoints. Tumor tissue was embedded and sectioned for further histological evaluation and immunostaining.

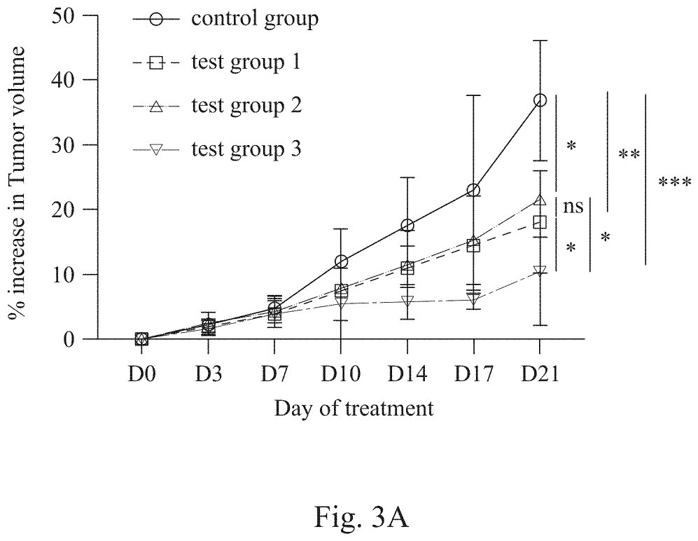

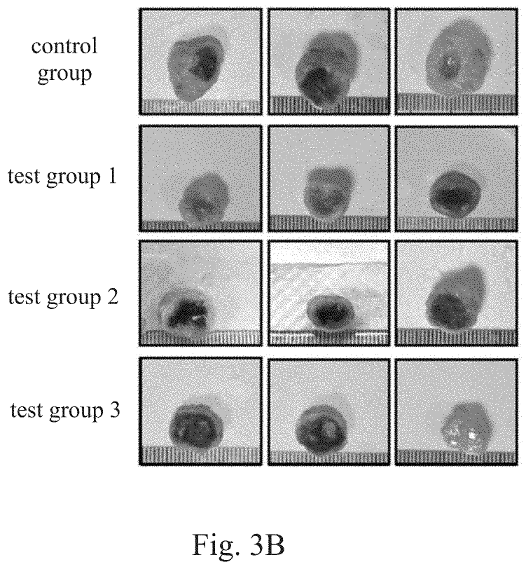

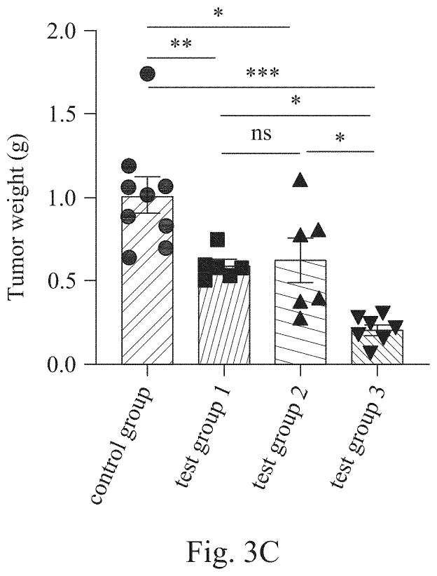

Please refer to FIGS. 3A to 3C, which show the analysis results of the effect of the xenogeneic mammary cell composition in the treatment of breast cancer. FIG. 3A shows the statistical results of tumor volume in different groups of mice during the experiment period, FIG. 3B shows photos of tumors in different groups of mice, and FIG. 3C is a statistical graph of tumor weights in different groups of mice. In FIGS. 3A and 3C, * indicates p<0.05; ** indicates p<0.01, and *** indicates p<0.001.

In FIGS. 3A to 3C, compared with the control group, the test group 1 treated with the xenogeneic mammary cell composition alone can significantly inhibit tumor growth, which can achieve similar effect as the test group 2 (treated with chemotherapy drug alone). The test group 3 treated with the xenogeneic mammary cell composition and chemotherapy drug at the same time had a more significant effect of inhibiting tumor growth.

II. The Effect of Xenogeneic Tissue Cell Composition in the Treatment of Kidney Cancer

To demonstrate the anti-kidney cancer effect of the xenogeneic tissue cell composition administered by the intralesional route, the xenogeneic renal cell composition isolated from porcine renal papillary tissue and expanded, and an immunocompetent orthotopic RAG-luc renal tumor mouse model is used to explore the efficacy thereof.

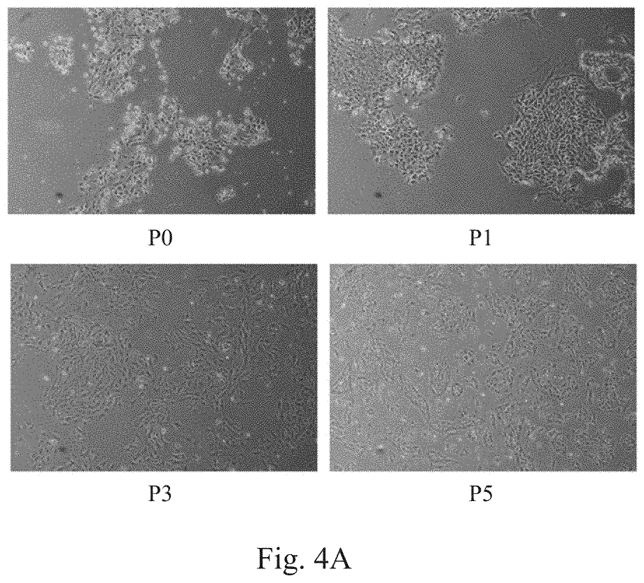

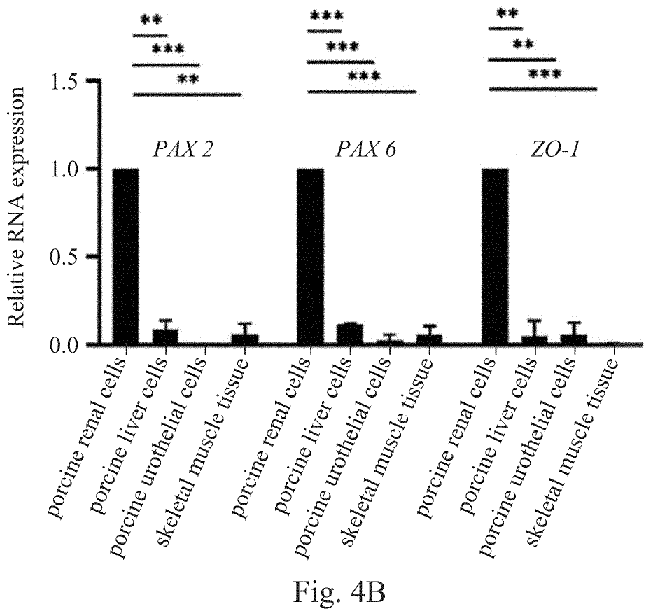

In the experiment, the isolation and expansion of the xenogeneic renal cell composition is performed first, and the cell characteristics of the isolated xenogeneic renal cell composition are analyzed. Please refer to FIGS. 4A and 4B, which show the analysis results of cell characteristics of the xenogeneic renal cell composition isolated from porcine kidney tissue. FIG. 4A shows photomicrographs of porcine renal cells of different passages, and FIG. 4B is the analysis result of expression analysis of related genes in porcine renal cells.

The results in FIG. 4A show that the isolated xenogeneic renal cell composition has the morphology of porcine renal epithelial cells. The results in FIG. 4B show that compared with porcine liver cells, porcine urothelial cells and skeletal muscle tissue, only porcine renal cells abundantly express renal cell-related genes such as PAX2 gene, PAX6 gene and ZO-1 gene, while other cells express none or a small amount of the renal cell-related genes. Statistical analysis in FIG. 4B was performed by one-way ANOVA, data are presented as mean±SD, ** indicates p<0.01, and *** indicates p<0.001.

A syngeneic animal model that is similar to kidney tumor and has a complete immune system is used to further evaluate the anti-kidney cancer effect of the xenogeneic renal cell composition in vivo. Therefore, an orthotopic RAG-luc renal tumor mouse model was established first. The RAG-luc renal cancer cell line (1×10⁶) derived from BALB/c mice and transfected with a luciferase reporter gene was injected into the kidney parenchyma of immunocompetent BALB/c mice, and the implanted RAG-luc renal cancer cell line developed into tumors. The model was established when tumor bioluminescent signal reached 10⁵ total plex and subsequent treatment was performed. There were 4 groups in the experiment, untreated control group, the test group 1 treated with the xenogeneic renal cell composition, the test group 2 treated with chemotherapy drug, and the test group 3 treated with both the xenogeneic renal cell composition and the chemotherapy drug, wherein the chemotherapy drug used in this experiment was Sunitinib, which is a tyrosine kinase inhibitor. The xenogeneic renal cell composition treated in test group 1 and test group 3 was intratumorally injected with 1×10⁶ xenogeneic renal cell composition on day 1 of the experiment. The chemotherapy drug treated in test group 2 and test group 3 was administered orally Sunitinib at 40 mg/kg daily. The treatment time was 4 weeks, and the tumor size was monitored with an IVIS imaging system during the experiment. The tumor-invaded kidney tissue was harvested and weighed when the mice died or reached the experiment endpoints.

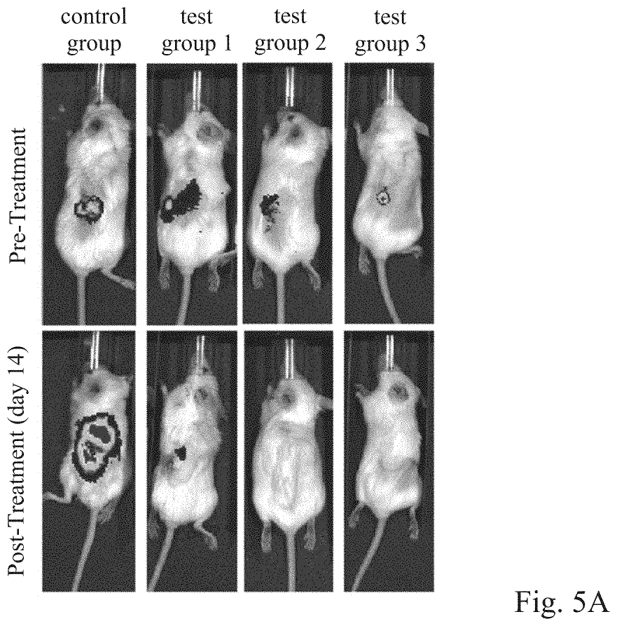

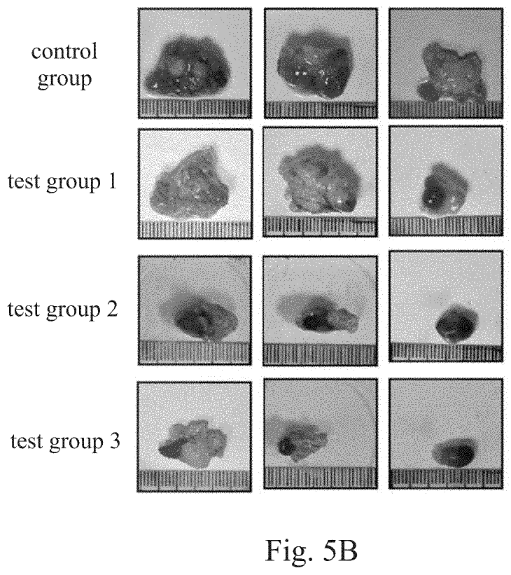

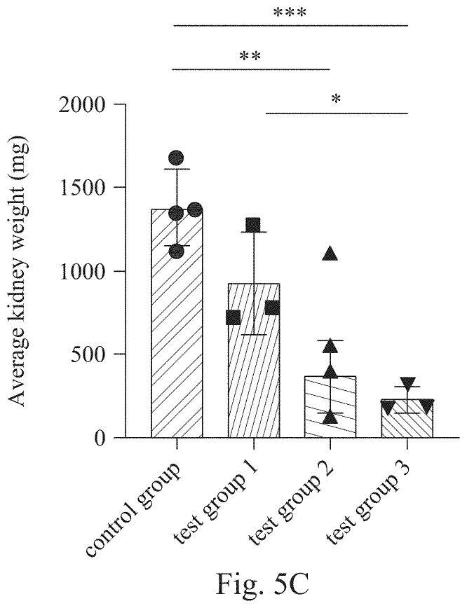

Please refer to FIGS. 5A to 5C, which show the analysis results of the effect of the xenogeneic renal cell composition in the treatment of kidney cancer. FIG. 5A shows the IVIS images of different groups of mice before treatment and after treatment to day 14, FIG. 5B shows photos of kidneys in different groups of mice, and FIG. 5C is a statistical graph of kidney weights in different groups of mice. In FIG. 5C, * indicates p<0.05; ** indicates p<0.01, and *** indicates p<0.001.

In FIGS. 5A to 5C, compared with the control group, the test group 1 treated with the xenogeneic renal cell composition alone can significantly inhibit tumor growth, which can achieve similar effect as the test group 2 (treated with chemotherapy drug alone). The test group 3 treated with the xenogeneic renal cell composition and chemotherapy drug at the same time had a more significant effect of inhibiting tumor growth. The results indicate that the xenogeneic renal cell composition can inhibit the progression of kidney tumor, and can be further combined with chemotherapy drug to achieve a better therapeutic effect.

III. The Effect of Xenogeneic Tissue Cell Composition in the Treatment of Liver Cancer

To demonstrate the anti-liver cancer effect of the xenogeneic tissue cell composition administered by the intralesional route, the xenogeneic liver cell composition isolated from porcine liver tissue and expanded, and an immunocompetent orthotopic Hepa 1-6-luc HCC liver tumor mouse model is used to explore the efficacy thereof.

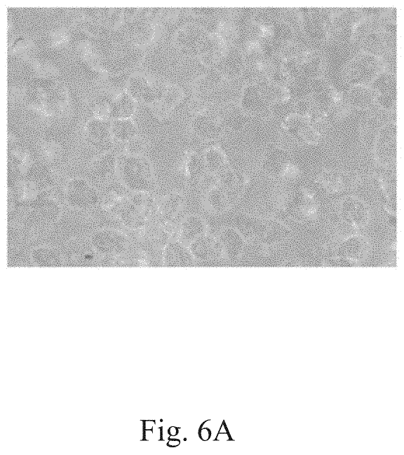

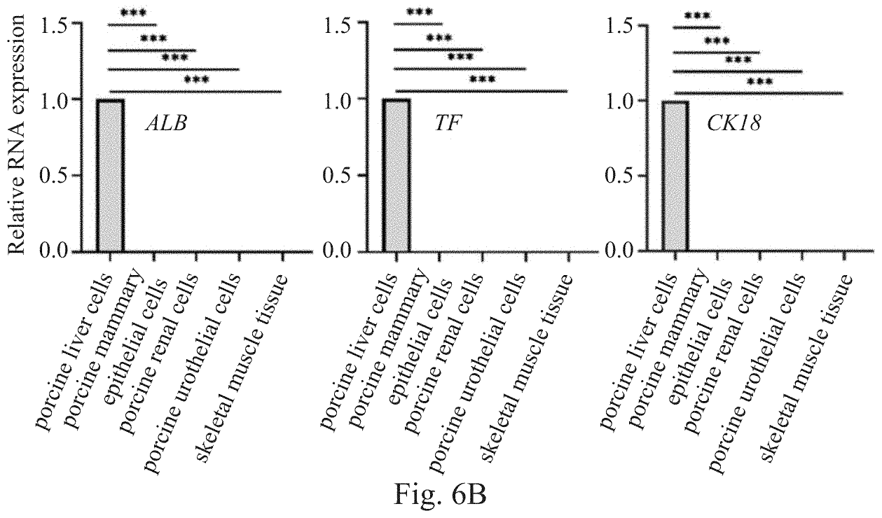

In the experiment, the isolation and expansion of the xenogeneic liver cell composition is performed first, and the cell characteristics of the isolated xenogeneic liver cell composition are analyzed. Please refer to FIGS. 6A and 6B, which show the analysis results of cell characteristics of the xenogeneic liver cell composition isolated from porcine liver tissue. FIG. 6A shows photomicrographs of porcine liver cells of different passages, and FIG. 6B is the analysis result of expression analysis of related genes in porcine liver cells.

The results in FIG. 6A show that the isolated xenogeneic liver cell composition has the morphology of primary porcine liver cells. The results in FIG. 6B show that compared with porcine mammary epithelial cells, porcine renal cells, porcine urothelial cells and skeletal muscle tissue, only porcine liver cells abundantly express liver cell-related genes such as ALB gene, TF gene and CK18 gene, while other cells do not express the liver cell-related genes. Statistical analysis in FIG. 6B was performed by one-way ANOVA, data are presented as mean±SD, and *** indicates p<0.001.

A syngeneic animal model that is similar to liver tumor and has a complete immune system is used to further evaluate the anti-liver cancer effect of the xenogeneic liver cell composition in vivo. Therefore, an orthotopic Hepa 1-6-luc liver tumor mouse model was established first. The Hepa 1-6-luc liver cancer cell line (1×10⁶) derived from C57BL6 mice and transfected with a luciferase reporter gene was injected into left lobes of immunocompetent C57BL6 mice, and the implanted Hepa 1-6-luc liver cancer cell line developed into tumors. The model was established when tumor bioluminescent signal reached 10⁵ total plex and subsequent treatment was performed. There were 4 groups in the experiment, untreated control group, the test group 1 treated with the xenogeneic liver cell composition, the test group 2 treated with chemotherapy drug, and the test group 3 treated with both the xenogeneic liver cell composition and the chemotherapy drug, wherein the chemotherapy drug used in this experiment was Sorafenib. The xenogeneic liver cell composition treated in test group 1 and test group 3 was intratumorally injected with 1×10⁶ xenogeneic liver cell composition on day 1 of the experiment. The chemotherapy drug treated in test group 2 and test group 3 was administered orally Sorafenib at 30 mg/kg five times a week. The treatment time was 2 weeks or 4 weeks, and the tumor size was monitored with an IVIS imaging system during the experiment. The tumor-invaded liver tissue was harvested and weighed when the mice died or reached the experiment endpoints.

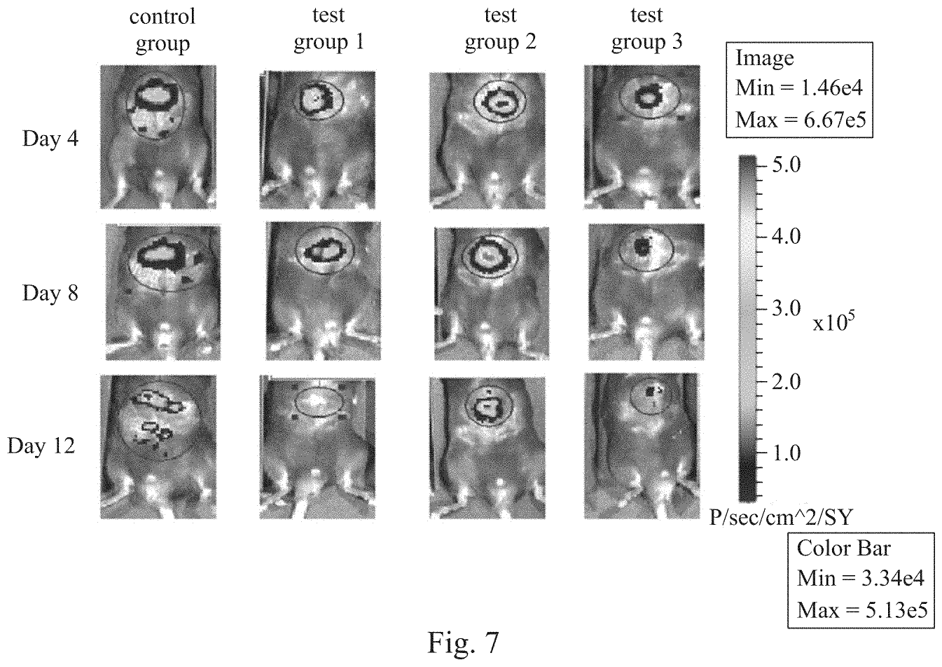

Please refer to FIG. 7 showing the analysis results of the effect of the xenogeneic liver cell composition in the treatment of liver cancer, which shows the IVIS images of different groups of mice from the treatment to day 4, day 8 and day 12. In FIG. 7, compared with the control group, the test group 1 treated with the xenogeneic liver cell composition alone can significantly inhibit tumor growth, which can achieve better effect than the test group 2 (treated with chemotherapy drug alone). The test group 3 treated with the xenogeneic liver cell composition and chemotherapy drug at the same time had a more significant effect of inhibiting tumor growth. The results indicate that the xenogeneic liver cell composition can inhibit the progression of liver tumor, and can be further combined with chemotherapy drug to achieve a better therapeutic effect.

IV. The Effect of Xenogeneic Tissue Cell Composition in the Treatment of Bladder Cancer

To demonstrate the anti-bladder cancer effect of the xenogeneic tissue cell composition administered by the intralesional route, the xenogeneic urothelial cell composition isolated from porcine urothelial tissue and expanded, and an immunocompetent orthotopic MBT-2 bladder tumor mouse model is used to explore the efficacy thereof.

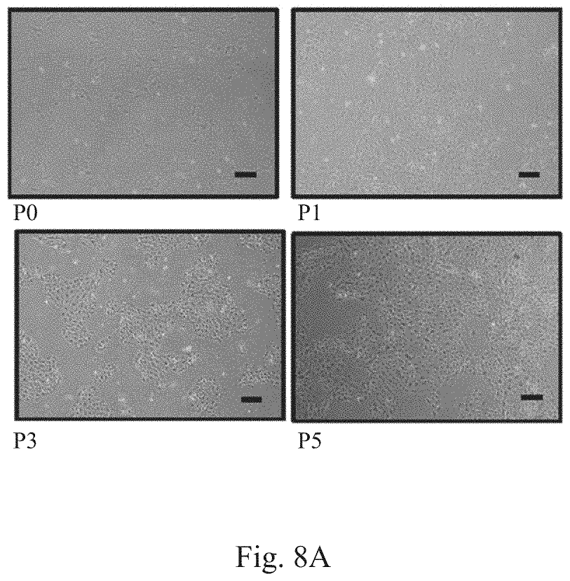

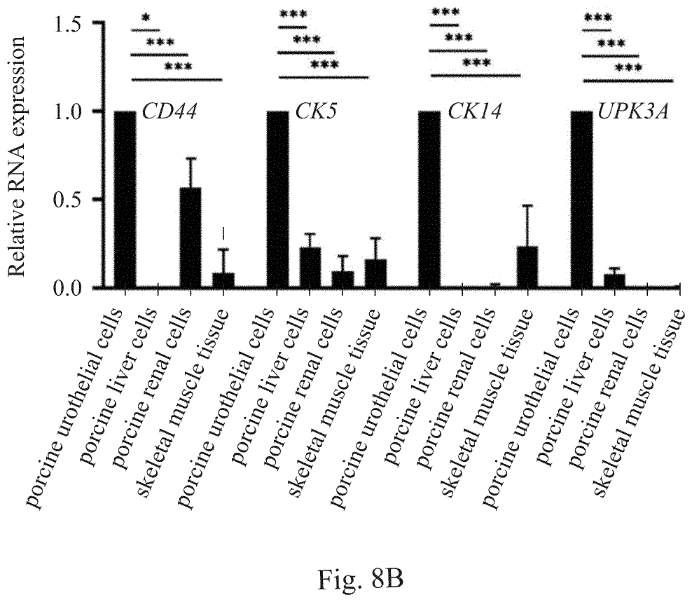

In the experiment, the isolation and expansion of the xenogeneic urothelial cell composition is performed first, and the cell characteristics of the isolated xenogeneic urothelial cell composition are analyzed. Please refer to FIGS. 8A and 8B, which show the analysis results of cell characteristics of the xenogeneic urothelial cell composition isolated from porcine. FIG. 8A shows photomicrographs of porcine urothelial cells of different passages, and FIG. 8B is the analysis result of expression analysis of related genes in porcine urothelial cells.

The results in FIG. 8A show that the isolated xenogeneic urothelial cell composition has the morphology of porcine urothelial cells. The results in FIG. 8B show that compared with porcine liver cells, porcine renal cells and skeletal muscle tissue, only porcine urothelial cells abundantly express urothelial cell-related genes such as CD44 gene, CK5 gene, CK14 gene and UPK3A gene, while other cells express none or a small amount of the urothelial cell-related genes. Statistical analysis in FIG. 8B was performed by one-way ANOVA, data are presented as mean±SD, and * indicates p<0.05, and *** indicates p<0.001.

A syngeneic animal model that is similar to bladder tumor and has a complete immune system is used to further evaluate the anti-bladder cancer effect of the xenogeneic urothelial cell composition in vivo. Therefore, an orthotopic MBT-2 bladder tumor mouse model was established first. The MBT-2 bladder cancer cell line (1×10⁶) derived from C3H/He mice was injected into the subcutaneous tissue of immunocompetent C3H/He mice, and the implanted MBT-2 bladder cancer cell line developed into tumors. The model was established when the tumor size reached to 50-100 mm³, and subsequent treatment was performed. There were 4 groups in the experiment, untreated control group, the test group 1 treated with the xenogeneic urothelial cell composition, the test group 2 treated with chemotherapy drug, and the test group 3 treated with both the xenogeneic urothelial cell composition and the chemotherapy drug, wherein the chemotherapy drugs used in this experiment were Gemcitabine and Cisplatin. The xenogeneic urothelial cell composition treated in test group 1 and test group 3 was intratumorally injected with 1×10⁶ xenogeneic urothelial cell composition on day 3 of the experiment and once a week for 2 weeks. The chemotherapy drug treated in test group 2 and test group 3 was intraperitoneally injected Gemcitabine at 6 mg/mouse on day 1 of the experiment, and intraperitoneal injected Cisplatin at 0.12 mg/mouse on day 2, once a week for 3 consecutive weeks. The tumor growth was assessed by measuring tumor volumes twice a week with a caliper and the volume is calculated using the formula: π/6×length×width². Tumor tissue was harvested and weighed when the mice died or reached the experiment endpoints.

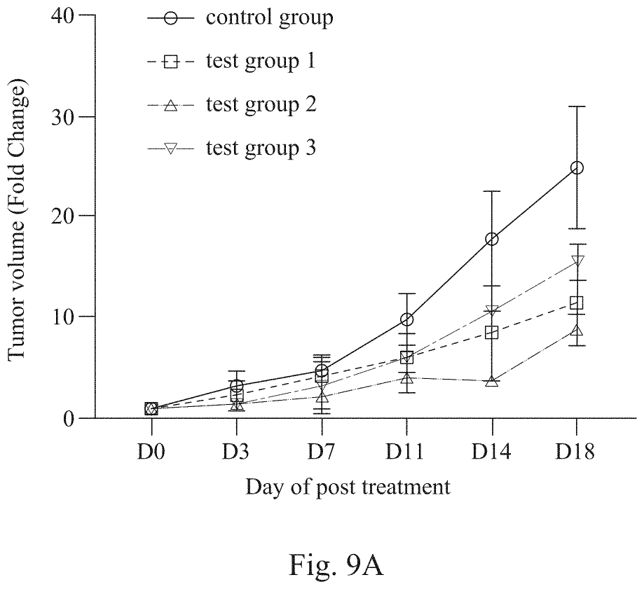

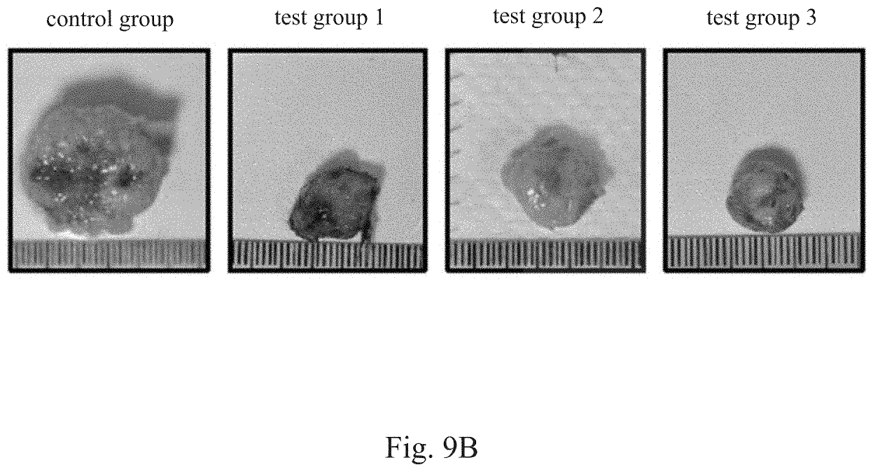

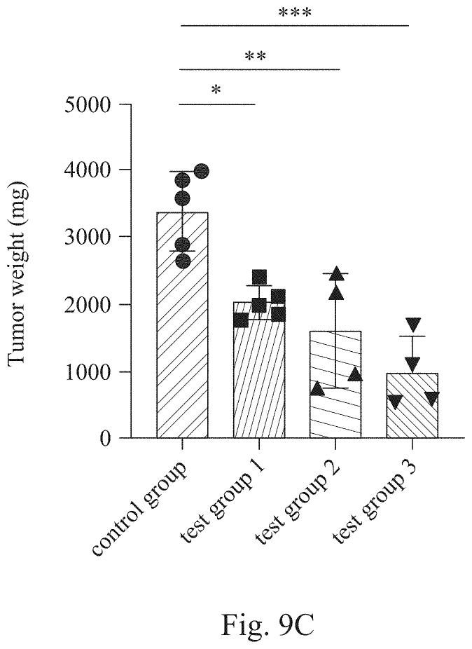

Please refer to FIGS. 9A to 9C, which show the analysis results of the effect of the xenogeneic urothelial cell composition in the treatment of bladder cancer. FIG. 9A shows the statistical results of tumor volume in different groups of mice during the experiment period, FIG. 9B shows photos of tumors in different groups of mice, and FIG. 9C is a statistical graph of tumor weights in different groups of mice. In FIG. 9C, * indicates p<0.05; ** indicates p<0.01, and *** indicates p<0.001.

In FIGS. 9A to 9C, compared with the control group, the test group 1 treated with the xenogeneic urothelial cell composition alone can significantly inhibit tumor growth, while the test group 3 treated with the xenogeneic urothelial cell composition and chemotherapy drug at the same time had a more significant effect of inhibiting tumor growth.

V. The Effect of Xenogeneic Tissue Cell Composition in the Treatment of Pancreatic Cancer

To demonstrate the anti-pancreatic cancer effect of the xenogeneic tissue cell composition administered by the intralesional route, the xenogeneic pancreatic cell composition isolated from porcine pancreatic tissue and expanded, and an immunocompetent heterotopic Pan 18 pancreatic tumor mouse model is used to explore the efficacy thereof.

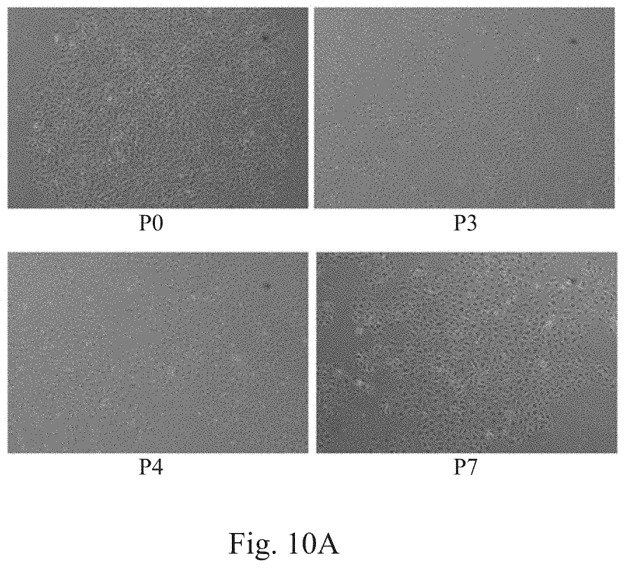

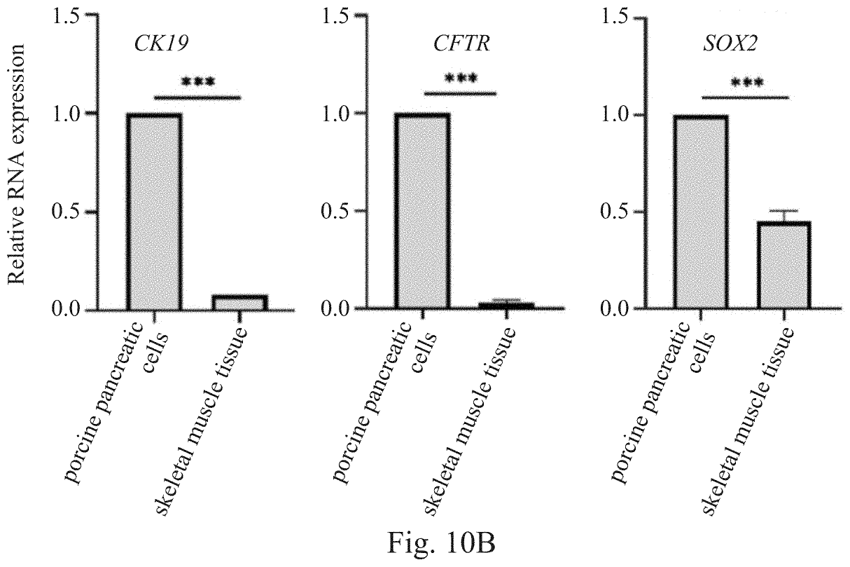

In the experiment, the isolation and expansion of the xenogeneic pancreatic cell composition is performed first, and the cell characteristics of the isolated xenogeneic pancreatic cell composition are analyzed. Please refer to FIGS. 10A and 10B, which show the analysis results of cell characteristics of the xenogeneic pancreatic cell composition isolated from porcine pancreatic tissue. FIG. 10A shows photomicrographs of porcine pancreatic cells of different passages, and FIG. 10B is the analysis result of expression analysis of related genes in porcine pancreatic cells.

The results in FIG. 10A show that the isolated xenogeneic pancreatic cell composition has the morphology of porcine pancreatic epithelial cells. The results in FIG. 10B show that compared with skeletal muscle tissue, only porcine pancreatic cells abundantly express pancreatic cell-related genes such as CK19 gene, CFTR gene and SOX2 gene, while skeletal muscle tissue expresses a small amount of the pancreatic cell-related genes. Statistical analysis in FIG. 10B was performed by one-way ANOVA, data are presented as mean±SD, and *** indicates p<0.001.

A syngeneic animal model that is similar to pancreatic tumor and has a complete immune system is used to further evaluate the anti-pancreatic cancer effect of the xenogeneic pancreatic cell composition in vivo. Therefore, a heterotopic Pan 18 pancreatic tumor mouse model was established first. The Pan 18 pancreatic cancer cell line (1×10⁶) derived from C57BL6 mice was injected into the dorsal side of immunocompetent C57BL6 mice, and the implanted Pan 18 pancreatic cancer cell line developed into tumors. The model was established when the tumor size reached to 50-100 mm³, and subsequent treatment was performed. There were 4 groups in the experiment, untreated control group, the test group 1 treated with the xenogeneic pancreatic cell composition, the test group 2 treated with chemotherapy drug, and the test group 3 treated with both the xenogeneic pancreatic cell composition and the chemotherapy drug, wherein the chemotherapy drug used in this experiment was Gemcitabine. The xenogeneic pancreatic cell composition treated in test group 1 and test group 3 was intratumorally injected with 1×10⁶ xenogeneic pancreatic cell composition on day 1 of the experiment. The chemotherapy drug treated in test group 2 and test group 3 was intraperitoneally injected Gemcitabine at 60 mg/kg once a week, and the treatment time was 3 weeks. The tumor growth was assessed by measuring tumor volumes twice a week with a caliper and the volume is calculated using the formula: π/6×length×width². Tumor tissue was harvested and weighed when the mice died or reached the experiment endpoints.

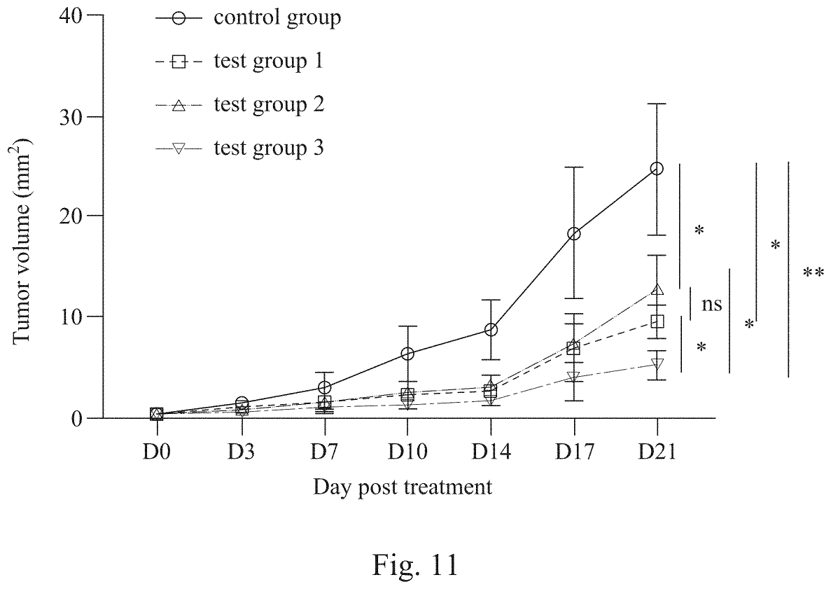

Please refer to FIG. 11 showing the analysis results of the effect of the xenogeneic pancreatic cell composition in the treatment of pancreatic cancer, which shows the statistical results of tumor volume in different groups of mice during the experiment period, wherein * indicates p<0.05, and ** indicates p<0.01. In FIG. 11, compared with the control group, the test group 1 treated with the xenogeneic pancreatic cell composition alone can significantly inhibit tumor growth, which can achieve similar effect as the test group 2 (treated with chemotherapy drug alone). The test group 3 treated with the xenogeneic pancreatic cell composition and chemotherapy drug at the same time had a more significant effect of inhibiting tumor growth.

In summary, the xenogeneic tissue cell composition of the present disclosure can be administered to tumor sites of similar or same histological type by the intralesional route to repair damaged tissues, restore the immune system to treat cancer. The experimental data indicate that the xenogeneic tissue cell composition of the present disclosure has excellent effect of inhibiting tumor progression in animal models such as breast cancer, kidney cancer, liver cancer, bladder cancer and pancreatic cancer. In addition, it can also be used in combination with another anticancer therapeutic agent, such as chemotherapy drug, to achieve synergistic effect, and has the potential to be used in the biomedical health care market.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

1. A method for treating a cancer of similar or same histological type, the method comprising administering a xenogeneic tissue cell composition to a subject in need for a treatment of the cancer of similar or same histological type, wherein the xenogeneic tissue cell composition does not comprise a tumor cell, and the xenogeneic tissue cell composition is administered to the cancer of similar or same histological type by an intralesional route.

2. The method of claim 1, wherein the intralesional route comprises an intratumoral administration and a peritumoral administration.

3. The method of claim 1, wherein the xenogeneic tissue cell composition comprises xenogeneic tissue-specific stem cells, xenogeneic tissue progenitor cells, xenogeneic tissue precursors and xenogeneic tissue mature cells.

4. The method of claim 1, wherein the xenogeneic tissue cell composition further comprises an extracellular matrix molecule and a polysaccharide.

5. The method of claim 1, wherein the xenogeneic tissue cell composition is isolated from a tissue of a mammal.

6. The method of claim 5, wherein the mammal is a human, a pig, a dog, a cat, a cow, a horse, a donkey, a deer, a goat, a sheep, a rabbit, a mouse, a rat, a guinea pig or a monkey.

7. The method of claim 1, wherein the xenogeneic tissue cell composition further comprises at least one chemotherapy drug.

8. The method of claim 7, wherein the at least one chemotherapy drug is selected from the group consisting of an alkylating agent, a nitrosourea agent, an antimetabolite, an antitumor antibiotic, an alkaloid derived from a plant, a topoisomerase inhibitor, a hormone therapy medicine, a hormone antagonist, an aromatase inhibitor, a P-glycoprotein inhibitor and a platinum complex derivative.

9. The method of claim 1, wherein the xenogeneic tissue cell composition further comprises a targeted therapy drug, an antibody drug, an immunomodulator or a combination thereof.

10. The method of claim 9, wherein the targeted therapy drug is selected from the group consisting of a tyrosine kinase inhibitor, a mitogen-activated protein kinase inhibitor, an anaplastic lymphoma kinase inhibitor, a B-cell lymphoma-2 inhibitor, a poly ADP ribose polymerase inhibitor, a selective estrogen receptor modulator, a phosphatidylinositol trikinase inhibitor, a Braf inhibitor, a cyclin-dependent kinase inhibitor and a heat shock protein 90 inhibitor.

11. The method of claim 9, wherein the antibody drug is selected from an antibody and an antibody drug complex.

12. The method of claim 9, wherein the immunomodulator is selected from a cytokine and an immune checkpoint inhibitor.
